(19) United States
(12) Patent Application Publication
(10) Pub. No.: US 2026/0096751 A1
(43) Pub. Date: Apr. 9, 2026

(54) WEIGHTED COMBINATION OF ANALYTE VALUES FOR MULTIPLE SENSING AREAS

(71) Applicant: Senseonics, Incorporated, Germantown, MD (US)

(72) Inventors: James Masciotti, Germantown, MD (US); Patricia Sanchez, Germantown, MD (US); Chad Hicks, Germantown, MD (US)

(73) Assignee: Senseonics, Incorporated, Germantown, MD (US)

(21) Appl. No.: 19/351,694

(22) Filed: Oct. 7, 2025

Related U.S. Application Data
(60) Provisional application No. 63/704,298, filed on Oct. 7, 2024.

(51) Int. Cl. A61B 5/145; G06F 17/18

(52) U.S. Cl. CPC A61B 5/14546; A61B 5/14503; A61B 5/1451; G06F 17/18

(57) ABSTRACT

An analyte monitoring system, which includes an analyte sensor, may perform a method including generating first, second, and third sets of one or more analyte values using first, second, and third measurement electronics, respectively, of first, second, and third sensing areas (SAs), respectively, of the analyte sensor. The first, second, and third SAs may be different. The method may include determining a distribution value based on a plurality of sets of analyte values including the first, second, and/or third sets of analyte values, and the distribution value may indicate a distribution of the plurality of sets of analyte values. The method may include determining a combined analyte level based at least on the plurality of sets of analyte values and the distribution value.

### CROSS-REFERENCE TO RELATED APPLICATION

The present application claims the benefit of priority to U.S. Provisional Application No. 63/704,298, filed Oct. 7, 2024, which is incorporated herein by reference in its entirety.

### BACKGROUND

### Field of Invention

This disclosure relates to weighted combination of analyte values for multiple sensing areas (SAs).

### Discussion of the Background

Analyte monitoring systems have been configured to generate analyte values using an implantable analyte sensor within a living body and determine a level (e.g., an amount and/or concentration) of an analyte (e.g., glucose) in the living body (e.g., in interstitial fluid or blood of the living body) based on the generated analyte values. However, certain challenges presently exist with respect to analyte monitoring. For example, in analyte monitoring systems having an analyte sensor including only a single sensing area (SA), the accuracy of the determined analyte level depends greatly on the performance of the single SA, which varies based on factors such as an extent to which analyte indicator molecules used by the sensing area have degraded, among other potential error causing issues. For another example, in analyte monitoring systems having an analyte sensor including multiple sensing areas, the analyte monitoring system may be configured to generate analyte values for each of the different SAs independently, and the amount and/or concentration of the analyte (a.k.a., “overall analyte level”) may be determined based on a combination of the analyte values for the different SAs. However, erroneous analyte values from one SA will reduce the accuracy of the determined overall analyte level. Thus, there is a need for improved analyte monitoring systems.

### SUMMARY

Aspects may relate to an analyte monitoring system having an analyte sensor including multiple sensing areas (SAs), and the analyte monitoring system may be configured to generate analyte values for each of the different SAs independently and to determine the amount and/or concentration of the analyte (a.k.a., “overall analyte level”) based on a combination of the analyte values for the different SAs. In scenarios where analyte values determined for one SA of the analyte sensor are substantially different from analyte values determined for other SAs of the analyte sensor, the diverged analyte values may be due to an error. The analyte monitoring system, in calculating the overall analyte level, may treat the diverged analyte values differently than the non-diverged analyte values. More specifically, the analyte monitoring system may de-weight the diverged analyte values (e.g., low quality measurements) in calculating the overall analyte level.

In one aspect, a method may be performed by an analyte monitoring system comprising an analyte sensor. The method may include generating a first set of one or more analyte values using first measurement electronics of a first sensing area (SA) of the analyte sensor. The method may include generating a second set of one or more analyte values using second measurement electronics of a second SA of the analyte sensor. The first and second SAs may be different. The method may include generating a third set of one or more analyte values using third measurement electronics of a third SA of the analyte sensor. The first, second, and third SAs may be different. The method may include determining (s508) a distribution value based on a plurality of sets of analyte values including the first, second, and/or third sets of analyte values. The distribution value may indicate a distribution of the plurality of sets of analyte values. The method may include determining a combined analyte level based at least on the plurality of sets of analyte values and the distribution value.

In some aspects, the first set of analyte values may include a plurality of analyte values generated for different instances of time using the first measurement electronics of the first SA of the analyte sensor, the second set of analyte values may include a plurality of analyte values generated for the different instances of time using the second measurement electronics of the second SA of the analyte sensor, and the third set of analyte values may include a plurality of analyte values generated for the different instances of time using the third measurement electronics of the third SA of the analyte sensor.

In some aspects, the method may include: determining whether the first set of analyte values satisfies a condition; determining whether the second set of analyte values satisfies the condition; determining whether the third set of analyte values satisfies the condition; and, based on determining that the first and second sets of analyte values do not satisfy the condition and the third set of analyte values satisfies the condition, not selecting the first and second SAs and selecting the third SA, wherein the combined analyte level is determined based on the selection of the third SA. In some aspects, the analyte sensor may include a plurality of SAs including the first, second, and third SAs, and the method may include generating the plurality of set of analyte values using measurement electronics of the plurality of SAs of the analyte sensor. In some aspects, determining the distribution value may include: calculating a first tendency value representing the third set of analyte values and calculating a second tendency value representing the plurality of sets of analyte values. In some aspects, whether the third set of analyte values satisfies the condition may be determined based at least on the first tendency value, the second tendency value, and the distribution value. In some aspects, the first tendency value may be one of a mean or a median of the third set of analyte values, the second tendency value may be one of a mean or a median of the plurality of sets of analyte values, and the distribution value may be one of a standard deviation, an interquartile range, or an interdecile range of the plurality of sets of analyte values.

In some aspects, determining whether the third set of analyte values satisfies the condition may include: calculating a comparative value based on the first tendency value, the second tendency value, and the distribution value; determining whether the comparative value is greater than a threshold value; and determining that the third set of analyte values satisfies the condition based on whether the comparative value is greater than the threshold value. In some aspects, the comparative value may be calculated as follows:

$$d = \frac{{C}_{1} \times {T}_{1} - {C}_{2} \times {T}_{2}}{{C}_{3} \times {D}_{2}}$$

where d is the comparative value, C₁ is a first constant, C₂ is a second constant, C₃ is a third constant, T₁ is the first tendency value, T₂ is the second tendency value, and D₂ is the distribution value.

In some aspects, if the third set of analyte values is determined to satisfy the condition, the distribution value may be determined not based on the third set of analyte values.

In some aspects, determining the combined analyte level may include: calculating a first SA analyte level for the first SA based on the first set of analyte values; determining a first weight for the first SA analyte level; calculating a second SA analyte level for the second SA based on the second set of analyte values; determining a second weight for the second SA analyte level; calculating a third SA analyte level for the third SA based on the third set of analyte values; and determining a third weight for the third SA analyte level. In some aspects, the combined analyte level may be determined based on the first SA analyte level, the first weight, the second SA analyte level, the second weight, the third SA analyte level, and the third weight. In some aspects, the first weight may be determined based at least on: the first SA analyte level, a tendency value representing the plurality of sets of analyte values, and the distribution value; the second weight may be determined based at least on: the second SA analyte level, the tendency value, and the distribution value; and the third weight may be determined based at least on: the third SA analyte level, the tendency value, and the distribution value.

In some aspects, the first weight may have a negative correlation with a difference between the first SA analyte level and the tendency value increases, the second weight may have a negative correlation with a difference between the second SA analyte level and the tendency value increases, and the third weight may have a negative correlation with a difference between the third SA analyte level and the tendency value increases. In some aspects, the tendency value may be one of a mean or a median of the plurality of sets of analyte values, and the distribution value may be one of a standard deviation, an interquartile range, or an interdecile range of the plurality of sets of analyte values. In some aspects, the first weight may be equal to

$$f ( \frac{{C}_{1} \times A {L}_{SA {1}^{- {C}_{2} \times CT}}}{{C}_{3} \times S} ) ,$$

the second weight may be equal to

$$f ( \frac{{C}_{1} \times A {L}_{SA {2}^{- {C}_{2} \times CT}}}{{C}_{3} \times S} ) ,$$

and the third weight may be equal to

$$f ( \frac{{C}_{1} \times A {L}_{SA {3}^{- {C}_{2} \times CT}}}{{C}_{3} \times S} ) ,$$

where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

In another aspect, an analyte monitoring system may be provided including an analyte sensor and a user device. The analyte monitoring system may be configured to generate a first set of one or more analyte values using first measurement electronics of a first sensing area (SA) of the analyte sensor and generate a second set of one or more analyte values using second measurement electronics of a second SA of the analyte sensor. The first and second SAs may be different. The analyte monitoring system may be configured to generate a third set of one or more analyte values using third measurement electronics of a third SA of the analyte sensor, and the first, second, and third SAs may be different. The analyte monitoring system may be configured to determine a distribution value based on a plurality of sets of analyte values including the first, second, and/or third sets of analyte values, and the distribution value may indicate a distribution of the plurality of sets of analyte values. The analyte monitoring system may be configured to determine a combined analyte level based at least on the plurality of sets of analyte values and the distribution value.

In some aspects, the first set of analyte values may include includes a plurality of analyte values generated for different instances of time using the first measurement electronics of the first SA of the analyte sensor, the second set of analyte values may include a plurality of analyte values generated for the different instances of time using the second measurement electronics of the second SA of the analyte sensor, and the third set of analyte values may include a plurality of analyte values generated for the different instances of time using the third measurement electronics of the third SA of the analyte sensor.

In some aspects, the analyte monitoring system may be configured to: determine whether the first set of analyte values satisfies a condition; determine whether the second set of analyte values satisfies the condition; determine whether the third set of analyte values satisfies the condition; and, based on determining that the first and second sets of analyte values do not satisfy the condition and the third set of analyte values satisfies the condition, not select the first and second SAs and selecting the third SA. In some aspects, the combined analyte level may be determined based on the selection of the third SA. In some aspects, the analyte sensor may include a plurality of SAs including the first, second, and third SAs, and the analyte monitoring system may be configured to generate the plurality of set of analyte values using measurement electronics of the plurality of SAs of the analyte sensor. In some aspects, determining the distribution value may include: calculating a first tendency value representing the third set of analyte values and calculating a second tendency value representing the plurality of sets of analyte values. In some aspects, whether the third set of analyte values satisfies the condition may be determined based at least on the first tendency value, the second tendency value, and the distribution value. In some aspects, the first tendency value may be one of a mean or a median of the third set of analyte values; the second tendency value may be one of a mean or a median of the plurality of sets of analyte values; and the distribution value may be one of a standard deviation, an interquartile range, or an interdecile range of the plurality of sets of analyte values.

In some aspects, determining whether the third set of analyte values satisfies the condition may include: calculating a comparative value based on the first tendency value, the second tendency value, and the distribution value; determining whether the comparative value is greater than a threshold value; and determining that the third set of analyte values satisfies the condition based on whether the comparative value is greater than the threshold value. In some aspects, the comparative value may be calculated as follows:

$$d = \frac{{C}_{1} \times {T}_{1} - {C}_{2} \times {T}_{2}}{{C}_{3} \times {D}_{2}}$$

where d is the comparative value, C₁ is a first constant, C₂ is a second constant, C₃ is a third constant, T₁ is the first tendency value, T₂ is the second tendency value, and D₂ is the distribution value.

In some aspects, if the third set of analyte values is determined to satisfy the condition, the distribution value may be determined not based on the third set of analyte values.

In some aspects, determining the combined analyte level may include: calculating a first SA analyte level for the first SA based on the first set of analyte values; determining a first weight for the first SA analyte level; calculating a second SA analyte level for the second SA based on the second set of analyte values; determining a second weight for the second SA analyte level; calculating a third SA analyte level for the third SA based on the third set of analyte values; and determining a third weight for the third SA analyte level. In some aspects, the combined analyte level may be determined based on the first SA analyte level, the first weight, the second SA analyte level, the second weight, the third SA analyte level, and the third weight. In some aspects, the first weight may be determined based at least on: the first SA analyte level, a tendency value representing the plurality of sets of analyte values, and the distribution value; the second weight may be determined based at least on: the second SA analyte level, the tendency value, and the distribution value; and the third weight may be determined based at least on: the third SA analyte level, the tendency value, and the distribution value. In some aspects, the first weight may have a negative correlation with a difference between the first SA analyte level and the tendency value increases; the second weight may have a negative correlation with a difference between the second SA analyte level and the tendency value increases; and the third weight may have a negative correlation with a difference between the third SA analyte level and the tendency value increases.

In some aspects, the tendency value may be one of a mean or a median of the plurality of sets of analyte values, and the distribution value may be one of a standard deviation, an interquartile range, or an interdecile range of the plurality of sets of analyte values. In some aspects, the first weight may be equal to

$$f ( \frac{{C}_{1} \times A {L}_{SA {1}^{- {C}_{2} \times CT}}}{{C}_{3} \times S} ) ,$$

the second weight may be equal to

$$f ( \frac{{C}_{1} \times A {L}_{SA 2} - {C}_{2} \times CT}{{C}_{3} \times S} ) ,$$

and the third weight may be equal to

$$f ( \frac{{C}_{1} \times A {L}_{SA 3} - {C}_{2} \times CT}{{C}_{3} \times S} ) ,$$

where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

Aspects of this disclosure provide a method and a system for determining the overall analyte level based on weighted averaging of the analyte values determined for different SAs according to the divergences of the analyte values, thereby improving the accuracy of the overall analyte level.

### BRIEF DESCRIPTION OF THE DRAWINGS

The accompanying drawings, which are incorporated herein and form part of the specification, illustrate various aspects.

FIG. 1 shows an analyte monitoring system according to some aspects.

FIG. 2 shows an implantable device according to some aspects.

FIG. 3A shows an analyte sensor including multiple sensing areas (SAs) according to some aspects.

FIGS. 3B-3E show samples (e.g., analyte values) generated by an analyte sensor for different SAs according to some aspects.

FIG. 4 shows a process according to some embodiments.

FIG. 5 shows a process according to some embodiments.

### DETAILED DESCRIPTION

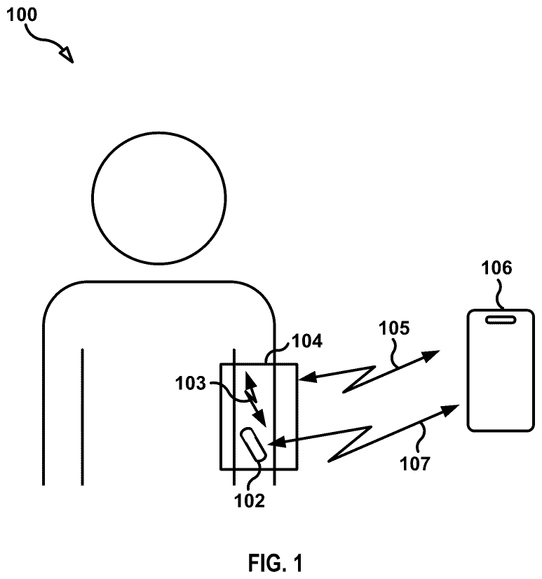

FIG. 1 shows a simplified view of an exemplary analyte monitoring system 100 (e.g., a glucose monitoring system) according to some aspects. In some aspects, as shown in FIG. 1, the analyte monitoring system 100 may include an analyte sensor 102, a transceiver 104, and/or a user device 106 (e.g., a smartphone). In some aspects, the user device 106 may be any electronic device that is capable of conveying information to a user. In some aspects, the user device 106 may convey information to a user by displaying the information on a display of the user device 106 and/or by generating audio indicating the information using speakers of the user device 106. Although the analyte monitoring system 100 is illustrated in FIG. 1 as including one analyte sensor 102, one transceiver 104, and one user device 106, this is not required, and, in some alternative aspects, the analyte monitoring system 100 may include more than one analyte sensor 102, more than one transceiver 104 (or no transceiver 104), and/or more than one user device 106.

In some aspects, the analyte sensor 102 may be a fully or partially implantable (e.g., subcutaneously implantable) analyte sensor. In some alternative aspects, the analyte sensor 102 may be a fully external analyte sensor. In some aspects, the analyte sensor 102 may be configured to generate measurements indicative of the existence, amount, and/or concentration of an analyte (e.g., glucose) in a medium (e.g., interstitial fluid) of a living animal (e.g., a living human). In some aspects, the measurements may include, for example and without limitation, light measurements, current measurements, and/or temperature measurements.

In some aspects, the analyte monitoring system 100 may generate analyte values indicating analyte levels for multiple sensing areas (SAs) of the analyte sensor 102 using measurements generated by the multiple SAs of the analyte sensor 102. In some aspects, the analyte sensor 102 may generate the analyte values using the measurements and convey the analyte values to the transceiver 104 via a communication channel 103 and/or to the user device 106 using a communication channel 107. In some aspects in which the analyte sensor 102 generates the analyte values, the analyte sensor 102 may additionally convey the measurements to the transceiver 104 via the communication channel 103 and/or to the user device 106 via the communication channel 107. Examples of the communication channels 103 and 107 include but are not limited to near field communication (NFC), Bluetooth, Wi-Fi, a wired connection, infrared light (IR) based communication, etc.

However, it is not required that the analyte sensor 102 generates the analyte values, and, in some alternative aspects, the transceiver 104 and/or the user device 106 may generate the analyte values using the measurements generated by the analyte sensor 102. In some aspects in which the analyte sensor 102 does not generate the analyte values, the analyte sensor 102 may convey only the measurements to the transceiver 104 and/or the user device 106.

In some aspects, the transceiver 104 may be an externally worn transceiver (e.g., attached via an armband, wristband, waistband, or adhesive patch). In some aspects, the transceiver 104 may be configured to remotely power and/or communicate with the analyte sensor 102 to receive the measurements and/or analytic values from the analyte sensor 102 via the communication channel 103.

In some aspects, the transceiver 104 may be configured to receive the measurements and/or analyte values from the analyte sensor 102. In some aspects in which the analyte sensor 102 or transceiver 104 generates the analyte values, the transceiver 104 may convey the analytic values to the user device 106 via a communication channel 105, and the transceiver 104 may also convey the measurements to the user device 106. In some aspects in which the user device 106 generates the analyte values, the transceiver 104 may be configured to forward the measurements generated by and received from the analyte sensor 102 to the user device 106, and the user device 106 generates the analyte values based on the received measurements. Examples of the communication channel 105 may include but are not limited to NFC, Bluetooth, Wi-Fi, wired connection, infrared light (IR) based communication, etc.

In some aspects in which the analyte sensor 102 or the transceiver 104 generates the analyte values based on the measurements generated by the analyte sensor 102, the user device 106 may be configured to receive the analyte values from the analyte sensor 102 and/or transceiver 104 via the communications channel 105 and/or 107, and the user device 106 may also receive the measurements generated by the analyte sensor 102. In some aspects in which the user device 106 generates the analyte values, the user device 106 may be configured to receive the measurements generated by the analyte sensor 102 directly from the analyte sensor 102 via the communications channel 107 and/or indirectly via the communications channels 103 and 105. In some aspects, the user device 106 may display analyte values on a screen and/or generate audio indicating analyte values using a speaker.

Although FIG. 1 shows the analyte sensor 102, the transceiver 104, and the user device 106 as three separate physical devices, in some aspects, two or all of the analyte sensor 102, the transceiver 104, and the user device 106 can be integrated into one device. For example, the transceiver 104 may be integrated into the analyte sensor 102. In another example, the transceiver 104 may be integrated into the user device 106. In such example, the user device 106 may be capable of communicating directly with the analyte sensor 102.

In some alternative aspects, one or more of the functions of the transceiver 104 and the user device 106 may be provided by the analyte sensor 102. For example, the analyte sensor 102 may be capable of performing measurements, processing the measurements, thereby generating the analyte values, and combining the analytic values to generate an overall analytic level. After generating the overall analytic level, the analyte sensor 102 may transmit the overall analytic level to the user device 106 such that the user device 106 can output the overall analytic level.

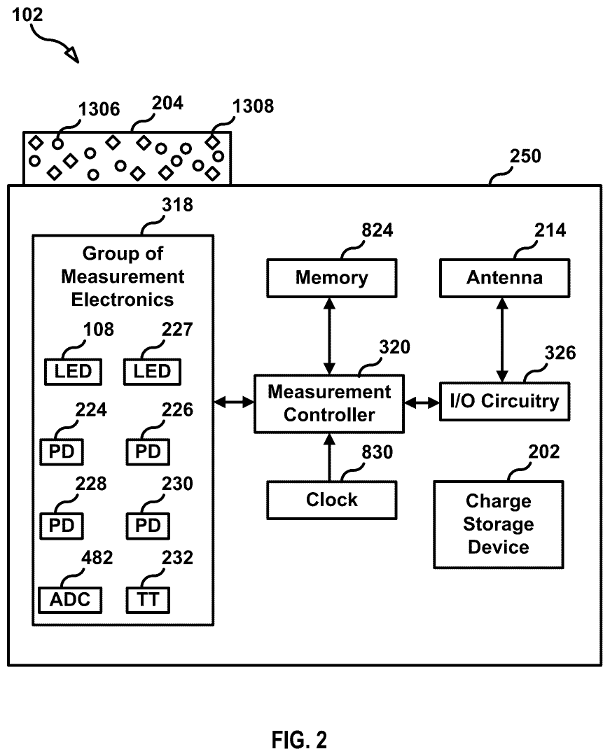

FIG. 2 illustrates an exemplary aspect in which the analyte sensor 102 of the system 100 is a fully implantable electro-optical sensor. However, this is not required, and, in some alternative aspects, the analyte sensor 102 may be a different type of analyte sensor (e.g., a transcutaneous electrochemical sensor) or a different type of apparatus (e.g., an insulin pump, a pacemaker, or electrical/heat therapy device). In some aspects, as shown in FIG. 2, the analyte sensor 102 may include a housing 250 (i.e., body, shell, capsule, or encasement), which may be rigid and biocompatible.

In some aspects, the housing 250 may be a silicon tube. However, this is not required, and, in other aspects, different materials and/or shapes may be used for the housing 250. In some aspects, the analyte sensor 102 may include a transmissive optical cavity (e.g., within the housing 250). In some aspects, the transmissive optical cavity may be formed from a suitable, optically transmissive polymer material, such as, for example, acrylic polymers (e.g., polymethylmethacrylate (PMMA)). However, this is not required, and, in other aspects, different materials may be used for the transmissive optical cavity.

In some aspects, the analyte sensor 102 may include analyte and/or interferent indicator material 204, which may be, for example, polymer grafts or hydrogels coated, diffused, adhered, embedded, or grown on or in one or more portions of the exterior surface of the housing 250. In some aspects, the analyte and/or interferent indicator material 204 may be porous and may allow the analyte (e.g., glucose) in a medium (e.g., interstitial fluid) to diffuse into the analyte and/or interferent indicator material 204.

In some aspects, as shown in FIG. 2, the analyte and/or interferent indicator material 204 may include analyte indicator molecules 1306 and/or interferent indicator molecules 1308 (e.g., degradation indicator molecules). In some aspects, the analyte sensor 102 may use the analyte indicator molecules 1306 to measure the presence, amount, and/or concentration of an analyte (e.g., glucose, oxygen, cardiac markers, low-density lipoprotein (LDL), high-density lipoprotein (HDL), or triglycerides).

In some aspects, the analyte sensor 102 may use the interferent indicator molecules 1308 to measure in vivo (e.g., ROS induced) signal degradation. In some aspects, in the analyte and/or interferent indicator material 204, the analyte indicator molecules 1306 and/or the interferent indicator molecules 1308 may be copolymerized into a single biocompatible hydrogel. In some aspects, the analyte indicator molecules 1306 and/or the interferent indicator molecules 1308 may have negligible spectral overlap and undergo similar degradation (e.g., similar degradation of boronic acids) in vivo.

In some aspects, the analyte indicator molecules 1306 may have one or more detectable properties (e.g., optical properties) that vary in accordance with (i) the amount or concentration of the analyte in proximity to the analyte and/or interferent indicator material 204 and (ii) an effect on the analyte indicator molecules 1306 (e.g., changes to the analyte indicator molecules 1306). In some aspects, the changes to the analyte indicator molecules 1306 may comprise the extent to which the analyte indicator molecules 1306 have degraded. In some aspects, the degradation may be (at least in part) ROS-induced oxidation. In some aspects, the analyte indicator molecules 1306 may be fluorescent analyte indicator molecules. In some aspects, the analyte indicator molecules 1306 may be distributed throughout the analyte and/or interferent indicator material 204. In some aspects, the analyte indicator molecules 1306 may be phenylboronic-based analyte indicator molecules. However, a phenylboronic-based analyte indicator is not required, and, in some alternative aspects, the analyte sensor 102 may include different analyte indicator molecules, such as, for example and without limitation, glucose oxidase-based indicators, glucose dehydrogenase-based indicators, and glucose binding protein-based indicators.

In some aspects, the interferent indicator molecules 1308 may have one or more detectable properties (e.g., optical properties) that vary in accordance with changes to the interferent indicator molecules 1308. In some aspects, the interferent indicator molecules 1308 are not sensitive to the amount of concentration of the analyte in proximity to the analyte and/or interferent indicator material 204. That is, in some aspects, the one or more detectable properties of the interferent indicator molecules 1308 do not vary in accordance with the amount or concentration of the analyte in proximity to the analyte and/or interferent indicator material 204. However, this is not required, and, in some alternative aspects, the one or more detectable properties of interferent indicator molecules 1308 may vary in accordance with the amount or concentration of the analyte in proximity to the analyte and/or interferent indicator material 204.

In some aspects, the changes to the interferent indicator molecules 1308 may comprise the extent to which the interferent indicator molecules 1308 have degraded. In some aspects, the degradation may be (at least in part) ROS-induced oxidation. In some aspects, the interferent indicator molecules 1308 may be fluorescent interferent indicator molecules. In some aspects, the interferent indicator molecules 1308 may be distributed throughout the analyte and/or interferent indicator material 204. In some aspects, the interferent indicator molecules 1308 may be phenylboronic-based interferent indicator molecules. However, phenylboronic-based interferent indicator molecules are not required, and, in some alternative aspects, the analyte sensor 102 may include different interferent indicator molecules 1308, such as, for example and without limitation, amplex red-based interferent indicator molecules, dichlorodihydrofluorescein-based interferent indicator molecules, dihydrorhodamine-based interferent indicator molecules, and scopoletin-based interferent indicator molecules.

In some aspects, the system 100 may use the interferent indicator molecules 1308 of the analyte and/or interferent indicator material 204, which may by sensitive to degradation by reactive oxygen species (ROS) but not sensitive to the analyte, to measure indirectly changes to the analyte indicator molecules 1306 of an analyte and/or interferent indicator material 204. In some aspects, the interferent indicator molecules 1308 may have one or more optical properties that change with extent of oxidation and may be used as a reference for measuring and correcting for extent of oxidation of the analyte indicator molecules 1306. In some aspects, the extent to which the interferent indicator molecules 1308 have degraded may correspond to the extent to which the analyte indicator molecules 1306 have degraded. For example, in aspects, the extent to which the interferent indicator molecules 1308 have degraded may be proportional to the extent to which the analyte indicator molecules 1306 have degraded. In some aspects, the extent to which the analyte indicator molecules 1306 have degraded may be calculated based on the extent to which the interferent indicator molecules 1308 have degraded. In some aspects, the system 50 may correct for changes in the analyte indicator molecules 1306 using an empiric correlation established through laboratory testing.

In some aspects, the analyte sensor 102 may include measurement electronics 318 (e.g., optical measurement electronics). In some aspects, the measurement electronics 318 may include one or more light sources and/or one or more photodetectors. For example, in some aspects, as shown in FIG. 2, the measurement electronics 318 may include one or more first light sources 108 that emit first excitation light over a wavelength range that interacts with the analyte indicator molecules 1306 in the analyte and/or interferent indicator material 204. In some aspects, the first excitation light may be ultraviolet (UV) light. In some aspects, the analyte sensor 102 may include one or more second light sources 227 that emit second excitation light over a wavelength range that interacts with the interferent indicator molecules 1308 in the analyte and/or interferent indicator material 204. In some aspects, the second excitation light may be, for example and without limitation, blue light.

In some aspects, the analyte indicator molecules 1306 may emit first emission light (e.g., fluorescent light) when irradiated by the first excitation light. In some aspects, an analyte (e.g., glucose) may bind reversibly to some of the analyte indicator molecules 1306, and the amount of first emission light emitted by an analyte indicator molecule 1306 may vary based on whether the analyte is bound to the analyte indicator molecule 1306. For example, when irradiated by the first excitation light, an analyte indicator molecule 1306 may emit a relatively large amount of first emission light if the analyte is bound to analyte indicator molecule 1306 and may emit a relatively small amount of first emission light 331 (or no first emission light 331) if analyte is not bound to the analyte indicator molecule 1306. Therefore, the amount of first emission light emitted by the analyte indicator molecules 1306 may vary based on the concentration of the analyte in proximity to the analyte and/or interferent indicator material 204. In some aspects, the amount of first emission light emitted by the analyte indicator molecule 1306 may also vary based on an amount of interference (e.g., the extent to which the analyte indicator molecules 1306 have degraded).

In some aspects, the interferent indicator molecules 1308 may emit second emission light (e.g., fluorescent light) when irradiated by the second excitation light. In some aspects, the amount of second emission light emitted by the interferent indicator molecules 1308 may vary based on an amount of interference (e.g., the extent to which the interferent indicator molecules 1308 have degraded). In some aspects, the amount of second emission light emitted by the interferent indicator molecules 1308 does not vary based on the concentration of the analyte in proximity to the analyte and/or interferent indicator material 204. In some aspects, degradation (e.g., oxidation) of the interferent indicator molecules 1308 may additionally or alternatively cause the absorption of the interferent indicator molecules 1308 (e.g., absorption of the second excitation light by the interferent indicator molecules 1308) to change.

In some aspects, as shown in FIG. 2, the measurement electronics 318 of the analyte sensor 102 may also include one or more photodetectors 224, 226, 228 (e.g., photodiodes, phototransistors, photoresistors, or other photosensitive elements). In some aspects, the measurement electronics 318 of the analyte sensor 102 may include one or more signal photodetectors 224 sensitive to first emission light (e.g., fluorescent light) emitted by the analyte indicator molecules 1306 such that a signal generated by a signal photodetector 224 is indicative of the level of first emission light of the analyte indicator molecules 1306 and, thus, the amount of analyte of interest (e.g., glucose). In some aspects, the measurement electronics 318 may include one or more reference photodetectors 226 sensitive to first excitation light that may be reflected from the analyte and/or interferent indicator material 204 such that a signal generated by a photodetector 226 in response thereto is indicative of the level of reflected first excitation light. In some aspects, the analyte sensor 102 may include one or more interferent photodetectors 228 sensitive to second emission light (e.g., fluorescent light) emitted by the interferent indicator molecules 1308 such that a signal generated by an interferent photodetector 228 in response thereto that is indicative of the level of second emission light of the interferent indicator molecules 1308 and, thus, the amount of degradation (e.g., oxidation). In some aspects, the one or more signal photodetectors 224 may be sensitive to second excitation light that may be reflected from the analyte and/or interferent indicator material 204. In this way, the one or more signal photodetectors 224 may act as reference photodetectors when the one or more second light sources 227 are emitting second excitation light.

However, it is not required that the one or more signal photodetectors 224 act as reference photodetectors when the one or more second light sources 227 are emitting second excitation light. In some alternative aspects, as shown in FIG. 2, the measurement electronics 318 of the analyte sensor 102 may include one or more second reference photodetectors 230 that act as reference photodetectors when the one or more second light sources 227 are emitting second excitation light. In some aspects, the one or more second reference photodetectors 230 may be sensitive to second excitation light that may be reflected from the analyte and/or interferent indicator material 204 such that a signal generated by a photodetector 230 in response thereto is indicative of the level of reflected second excitation light.

In some aspects, one or more of the photodetectors 224, 226, 228, 230 may be covered by one or more filters that allow only a certain subset of wavelengths of light to pass through and reflect (or absorb) the remaining wavelengths. In some aspects, one or more filters on the one or more signal photodetectors 224 may allow only a subset of wavelengths corresponding to first emission light and/or the reflected second excitation light. In some aspects, one or more filters on the one or more reference photodetectors 226 may allow only a subset of wavelengths corresponding to the reflected first excitation light. In some aspects, one or more filters on the one or more interferent photodetectors 228 may allow only a subset of wavelengths corresponding to second emission light. In some aspects in which the analyte sensor 102 includes one or more second reference photodetectors 230, one or more filters on the one or more second reference photodetectors 230 may allow only a subset of wavelengths corresponding to the reflected second excitation light.

In some aspects, as shown in FIG. 2, the measurement electronics 318 of the analyte sensor 102 may include one or more temperature transducers 232. In some aspects, the measurement electronics 318 may include one or more light source drivers, one or more amplifiers, one or more analog-to-digital convertors (ADCs) 482, one or more comparators, and/or one or more multiplexors. In some aspects, the one or more ADCs 482 may convert analog signals output by the photodetectors 224, 226, 228, 230 and/or one or more temperature transducers 232 to digital signals.

In some aspects, as shown in FIG. 2, the analyte sensor 102 may include a charge storage device 202, a measurement controller 320, a memory 824, a clock 830, input/output (I/O) circuitry 326, and/or an antenna 214. In some aspects, the I/O circuitry 326 may include I/O digital circuitry 334 and/or I/O analog circuitry (see FIG. 2E). In some aspects, the antenna 214 may be electrically connected to the I/O circuitry 326, which may use current flowing through the antenna 214 to generate power for the analyte sensor 102 and/or to extract data from the current. In some aspects, the I/O circuitry 326 may also convey data (e.g., to the transceiver 101 and/or display device 105) by modulating the current flowing through the antenna 214. In some aspects, the I/O circuitry 326 may (at least at times) be electrically connected to and powered by the charge storage device 202.

In some aspects, when electrically connected to and powered by the charge storage device 202, the clock 830 may provide a continuous clock for driving circuitry of the analyte sensor 102 (e.g., even when the analyte sensor 102 is not receiving power from an external device such as the transceiver 101 and/or the display device 105). In some aspects, the measurement controller 320 may be a computer. In some aspects, the analyte sensor 102 may use the continuous clock output of the clock 830 to keep track of time and initiate autonomous, self-powered analyte measurements when appropriate (e.g., at periodic intervals, such as, for example, every minute, every two minutes, every 5 minutes, every 10 minutes, every 15 minutes, every half-hour, every hour, every two hours, every six hours, every twelve hours, or every day). In some aspects, the measurement controller 320 may control the measurement electronics 318 to perform an autonomous analyte measurement sequence, and the results of the autonomous analyte measurement may be stored in the memory 824. The autonomous analyte measurements may be stored in the memory 824. In some aspects, the I/O circuitry 326 may convey one or more of the stored measurements to the external device (e.g., the transceiver 101 and/or the display device 105) at a later time. For example, in some request aspects, the I/O circuitry 326 may convey one or more of the stored measurements in response to the analyte sensor 102 receiving and decoding a measurement data request from the transceiver 101 and/or the display device 105. In some alternative aspects, the I/O circuitry 326 may convey one or more of the stored measurements in response to detecting that the transceiver 101 and/or display device 105 is present (e.g., when an electrodynamic field generated by the transceiver 101 and/or display device 105 induces a current in the antenna 214 of the analyte sensor 102).

In some aspects, the memory 824 may be a nonvolatile storage medium. In some aspects, the memory 824 may be an electrically erasable programmable read only memory (EEPROM). However, in some alternative aspects, other types of nonvolatile storage media, such as flash memory, may be used. In some aspects, the memory 824 may include an address decoder. In some aspects, the memory 824 may store measurement information autonomously generated while the analyte sensor 102 is powered from the charge storage device 202. In some aspects, the memory 824 may additionally or alternatively store one or more time-stamps identifying when the measurement data was generated, sensor calibration data, a unique sensor identification, setup information, and/or integrated circuit calibration data. In some aspects, the unique identification information may, for example, enable full traceability of the analyte sensor 102 through its production and subsequent use.

In some aspects, as shown in FIG. 2, the analyte sensor 102 may include one sensing device, which may include the group of measurement electronics 318 that interact with (e.g., emits excitation light to and detects light reflected and/or emitted by) the analyte and/or interferent indicator material 204. In some aspects, the measurement electronics 318 that interact with the analyte and/or interferent indicator material 204 may be disposed in a particular area of the analyte sensor 102. In this disclosure, the area where the measurement electronics 318 of the analyte sensor 102 is disposed is referred to as a sensing area (SA). Even though FIG. 2 shows only one sensing device and only one SA, in some alternative aspects, the analyte sensor 102 may include more than one sensing SA (e.g., two, three, four, five, or ten SAs), which may be part of one or more sensing devices (e.g., two, three, four, five, or ten sensing devices).

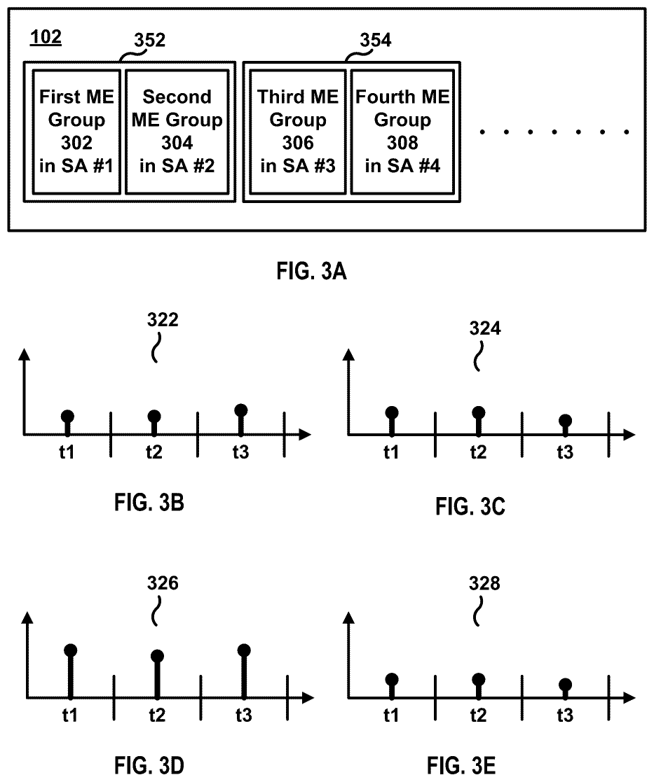

For example, as shown in FIG. 3A, the analyte sensor 102 may include first and second sensing devices 352 and 354, and each of the first and second sensing devices 352 and 354 may include two SAs for a total of four SAs for the analyte sensor 102. However, the number of sensing devices included in the analyte sensor 102 is not limited to two, and the analyte sensor 102 may include any number of sensing devices that is greater than or equal to one, such as, for example, one, three, four, five, or ten sensing devices. Similarly, the number of SAs in a sensing device is not limited to two, and a sensing device of the analyte sensor 102 may include any number of SAs that is greater than or equal to one, such as, for example, one, three, four, five, or ten SAs.

In some aspects, as shown in FIG. 3A, the sensing devices 352 and 354 may each include one or more groups of measurement electronics (“MEs”) (e.g., 318 shown in FIG. 2) that interacts with analyte and/or interferent indicator material 204 on a portion of the exterior surface of the housing 250. In some aspects, the sensing devices 352 and 354 may share a charge storage device 202 and/or an antenna 214. That is, in some aspects in which the analyte sensor 102 includes multiple sensing devices, the antenna 214 may be electrically connected to the circuitry of the multiple sensing devices (e.g., sensing devices 352 and 354), and the charge storage device 202 may be connected to the circuitry of the multiple sensing devices.

In some aspects, as further shown in FIG. 3A, in some aspects, each of the sensing devices 352 and 354 may include more than one group of MEs. For example, as shown in FIG. 3A, the sensing device 352 may include a first ME group 302 in a sensing area (SA) #1 and a second ME group 304 in SA #2. Similarly, the sensing device 354 may include a third ME group 306 in SA #3 and a fourth ME group 308 in SA #4. The SAs #1-4 are four different areas of the analyte sensor 102 where the ME groups 302-308 are disposed. As noted above, even though FIG. 3A shows that the number of SAs (i.e., the number of ME groups) included in each sensing device is two, in some alternative aspects, the number of SAs in each sensing device may be a different number.

In some aspects, each of the ME groups 302-308 may include one or more of the elements 108, 224, 226, 227, 228, 230, 232, and/or 482 shown in FIG. 2. In some aspects, each of the ME groups 302-308 may include the elements 108, 224, 226, 227, 228, 230, 232, and/or 482. In some alternative aspects, the ME groups 302-308 may share some of the elements 108, 224, 226, 227, 228, 230, 232, and/or 482. More specifically, in one example, the first ME group 302 may include the elements 108, 224, 226, 227, 228, 230, 232, and 482, but the second ME group 304 may only include the elements 108, 224, 226, 227, 228, and 230. In this example, the ME groups 302 and 304 may share the use of the ADC 482 and the temperature transducer 232.

In some aspects, as shown in FIG. 3A, in some aspects, the analyte sensor 102 may include more than one ME groups for generating measurements that may be used for determining analyte values. For example, as shown in FIG. 3A, the sensing device 352 may include the four ME groups 302, 304, 306, and 308 for generating measurements for the four SAs #1-#4, respectively.

In some aspects, each ME group may be configured to generate a set of measurements for an SA, and the measurements generated from the ME groups may be used to generate analyte values for the SAs. In some aspects, the generated analyte values for the SAs may be combined to calculate an overall analyte level. More specifically, in one example, each ME group included in the analyte sensor 102 may generate measurements, and the analyte sensor 102 may send the measurements to the transceiver 104. Based on the received measurements, the transceiver 104 may calculate analyte values for the SAs, combine the calculated analyte values for the SAs to calculate an overall analyte level, and send the calculated overall analyte value to the user device 106. The user device 106 may output (e.g., display or generate a sound) the received overall analyte level.

However, as illustrated in FIGS. 3B-3E, there may be a scenario where the set of measurements generated from one of the ME groups is diverged from the remaining sets of measurements generated from other ME groups. FIG. 3B shows a set 322 of analyte values generated based on measurements generated by the first ME group 302 in the SA #1. FIG. 3C shows a set 324 of analyte values generated based on measurements generated by the second ME group 304 in the SA #2. FIG. 3D shows a set 326 of analyte values generated based on measurements generated by the third ME group 306 in the SA #3. FIG. 3E shows a set 328 of analyte values generated based on measurements generated by the fourth ME group 308 in the SA #4. The analyte values included in each set are generated at different timings. For example, the first analyte value in each set is generated at t1, the second analyte value in each set is generated at t2, and the third analyte value in each set is generated at t3. In other words, each set of analyte values is generated during a time window of N (here N=3). Here, the time window of N means a time interval comprising N number of sub-intervals having the same time length. Even though FIGS. 3B-3E show that N is equal to three, N can be any number that is greater than 0. In other words, the number of analyte values included in each set may be any number that is greater than 0 (e.g., N may be equal to one, two, four, five, eight, ten, fifteen, or twenty analyte values).

As shown in FIGS. 3B-3E, the third set 326 of analyte values generated based on measurements generated by the third ME group 306 in the SA #3 is diverged from the remaining sets 322, 324, and 328 of analyte values generated based on measurements generated by other ME groups. In this scenario, it may be desirable to exclude or give less weight to the third set 326 of analyte values in calculating the combined analyte level. Thus, according to some aspects, the contribution of a set of analyte values to the combined analyte level may be adjusted based on the divergence of the set of analyte values with respect to other sets of analyte values.

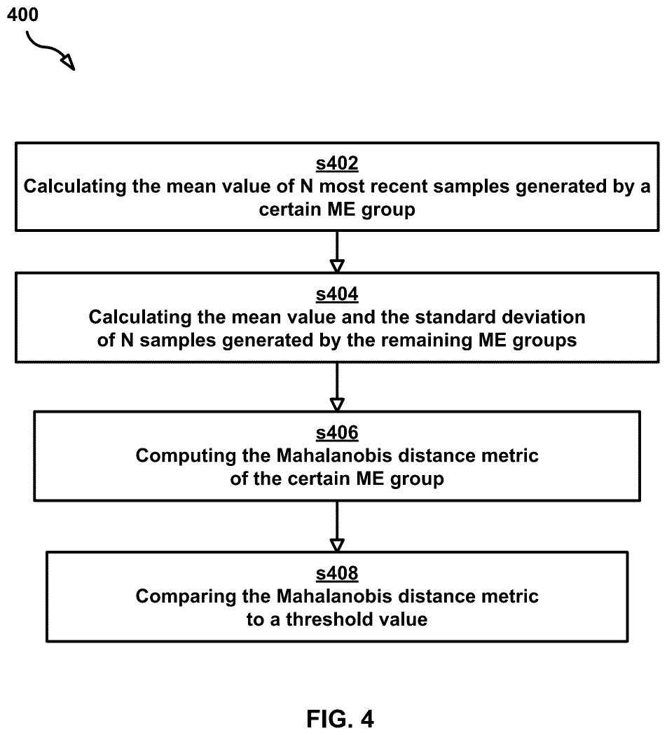

FIG. 4 shows one exemplary process 400 for determining whether a set of analyte values generated based on measurements by an ME group associated with an SA (i.e., a set of analyte values generated for an SA) is diverged from other sets of analyte values generated for other SAs, according to some aspects. In some aspects, the analyte monitoring system 100 (e.g., the analyte sensor 102, transceiver 104, and/or user device 106 of the analyte monitoring system 100) may perform one or more steps of the process 400. In some aspects, the transceiver 104 may perform one or more steps of the process 400, and, in some alternative aspects, the analyte sensor 102 or the user device 106 may performed one or more steps of the process 400. In some aspects, the process 400 may be performed for each of the ME group 302-308.

In some aspects, as shown in FIG. 4, the process 400 may include a step 402. The step 402 may include calculating the mean value (meanₐ) of N most recent samples (a.k.a., “analyte values”) generated by a certain ME group. For example, the step 402 may include calculating the mean value of three samples (i.e., N=3) from the set 326 of analyte values generated based on measurements by the third ME group 306 in the SA #3.

In some aspects, as shown in FIG. 4, the process 400 may include a step 404. The step 404 may include calculating the mean value (mean_{b}) and the standard deviation (std_{b}) of N samples generated based on measurements by the remaining ME groups. For example, the step 404 may include calculating the mean value and the standard deviation of nine samples from the sets 322, 324, and 328 of analyte values generated based on measurements by the ME groups 302, 304, and 308. Although FIG. 4 shows that the step 404 is performed after the step 402, in some alternative aspects, the step 402 may be performed after or simultaneously with the step 404.

In some aspects, as shown in FIG. 4, the process 400 may include a step 406. The step 406 may include computing the Mahalanobis distance metric of the certain ME group (e.g., the third ME group 306 in the SA #3) from the remaining ME groups. In some aspects, the Mahalanobis distance metric (dₐ) may be calculated as follows:

$${d}_{a} = \frac{{C}_{1} \times {mean}_{a} - {C}_{2} \times {mean}_{b}}{{C}_{3} \times {std}_{b}}$$

where each of C₁, C₂, and C₃ is a constant.

In some aspects, mean_{b} and std_{b} may be calculated based not only on the samples from the ME groups included in an analyte sensing device but also on the samples from the ME groups included in two or more analyte sensing devices included in the device 102.

In some aspects, as shown in FIG. 4, the process 400 may include a step 408. The step 408 may include comparing the Mahalanobis distance metric to a threshold value. If the Mahalanobis distance metric is greater than or equal to the threshold value, the certain ME group may be determined to be diverged from other ME groups. In contrast, if the Mahalanobis distance metric is less than the threshold value, the certain ME group may be determined to be not diverged from other ME groups.

In some alternative aspects, instead of the standard deviation (SD), another distribution parameter may be used. For example, in some alternative aspects, either an interquartile range (IQR) or an interdecile range (IDR) may be used instead.

In some aspects, after determining that a certain ME group is diverged from other ME groups, the Mahalanobis distance metric of the other ME groups may be calculated or recalculated by excluding the samples generated by the diverged ME group (i.e., outlier). For example, if the third ME group 306 is determined to be diverged, the Mahalanobis distance metric of the first ME group 302 may be calculated as follows:

$${d}_{302} = \frac{{mean}_{302} - {mean}_{304 , 308}}{{std}_{304 , 308}}$$

where mean₃₀₂ is the mean value of the three samples from the set 322 of analyte values generated based on measurements by the first ME group 302, mean_{304,308} is the mean value of the six samples from the sets 324 and 328 of the second and fourth ME groups 304 and 308, and std_{304,308} is the standard deviation of the six samples from the sets 324 and 328 of the second and fourth ME groups 304 and 308. Here, the samples of the diverged ME group (i.e., the third ME group 306) may be excluded from calculating the mean value and the standard deviation.

An example of the computer code implementing the steps of the process 400 is shown below.

```
class Distances(BaseFeature):
  “‘
    Mahalanobis Distance of last window value to others
    Cross channel divergence metric
  ”’
  def _{——}init_{——}(self, columns=‘all’, window_sizes=[10]):
    “‘
    Args:
      columns (str or list, optional): If string, should be ‘all’. If list, then
          is a list of columns names to which the feature functionality should be applied.
          Some features use specific columns and ignore this. Defaults to ‘all’.
      window_size (list, int or None, optional): If None, expect feature to
            not use a sliding window in its processing. If an int or list of int,
            feature can apply its methods to sliding windows of those sizes. Defaults to
            None.
    Argument info available to all features; whether/how they use the info or not is their
choice.
    ”’
    super( )._{——}init_{——}(columns, window_sizes)
#**************************************************************************
  def generate(self, am):
    “‘
    Args:
      am (DataFrame): Analysis matrix.
    Returns:
      DataFrame: New feature data.
    ”’
    # Set up groups of columns across which to compute divergence
    matrix = am if self.columns == ‘all’ else am[self.columns]
    cols = du.group_columns(matrix.columns)
    col_groups = [d for c in cols.values( ) if isinstance(c, dict) for d in c.values( )]
    col_flat = [j for i in col_groups for j in i]
    output_names = [(self.get_class( ) + ‘:zScore(‘ + i + ’,’, self.get_class( ) + ‘:zScoreInstant(‘ +
i + ’,’) for i in col_flat]
    output_flat = [j for i in output_names for j in i]
    # Compute the divergence of each channel within each group
    div_fnc = partial(self._divergence, column_groups=col_groups)
    div_data = _slide_window(matrix, self.window_sizes, ‘extend’, div_fnc, output_flat)
    return div_data
#**************************************************************************
  def _divergence(self, am, column_groups):
    “‘
    Args:
      am (DataFrame): Analysis matrix.
    Returns:
      list: New row of feature data.
    ”’
    results = [ ]
    for col_group in column_groups:
      if len(col_group) > 1:
        for c in col_group:
          # Compute Mahalanobis distance from test channel to other channels in group
          background_columns = [i for i in col_group if i != c]
          background_data = am[background_columns].to_numpy( )
          background_mean = np.mean(background_data, axis=1) if len(background_data)
> 0 else 1
          foreground_data = am[c].to_numpy( )
          mean_delta = np.mean(np.abs(foreground_data − background_mean))
          std_background = np.std(background_data.flatten( ))
          if std_background > 0:
            results.append(mean_delta / std_background) # z_score
          else:
            results.append(0.0)
      else:
        results.append(0.0)
    return results
```

After identifying a diverged ME group (e.g., the third ME group 306), an overall analyte level may be determined based on a weighted combination of the analyte values generated by the ME groups. For example, the overall analyte level (ALₒᵥₑᵣₐₗₗ) may be calculated as follows:

$$A {L}_{overall} = {\sum }_{a = 1}^{M} {w}_{a} \times A {V}_{a}$$

where ALₒᵥₑᵣₐₗₗ is the overall analyte level, M is the number of ME groups/SAs, wₐ is a weight value assigned to a certain ME group, and AVₐ is an analyte value generated by a certain ME group.

In some aspects, wₐ of a certain ME group may be a function of the Mahalanobis distance metric (d_{M}) of the certain ME group—i.e., wₐ=f(d_{M}). In one example,

$${w}_{a} = {C}_{4} \times \frac{1}{{d}_{M}}$$

where C₄ is a constant and d_{M} is the Mahalanobis distance metric. In these aspects, the more a certain ME group is diverged from the rest of the ME groups, less weight is given to the diverged ME group, and thus less contribution is made by the samples of the diverged ME group to the overall analyte level.

In some aspects, once a certain ME group is determined to be diverged from other ME groups, the samples generated by the diverged ME group may be excluded from being used in calculating the overall analyte level.

Alternatively, in some aspects, wₐ of a certain ME group (e.g., the third ME group 306) may set to be a function of 1) a difference between an analyte value generated from the certain ME group and a central tendency value (CT) (e.g., mean, median, bi-mean, weighted mean) of the analyte values generated from the remaining ME groups (e.g., the ME groups 302, 304, and 308) or all ME groups (e.g., the ME groups 302, 304, 306, and 308) and 2) a measure of spread (S) (e.g., SD, IQR, IDR) of the analyte values generated from the remaining ME groups or all ME groups. For example,

$${w}_{a} = f ( \frac{{C}_{5} \times A {V}_{a} - {C}_{6} \times CT}{{C}_{7} \times S} )$$

where each of C₅, C₆, and C₇ is a constant.

The following paragraphs describe detailed exemplary methods of calculating the overall analyte level according to some aspects. In the paragraphs below, glucose is used as an example of the analyte.

In some aspects, the final glucose level (i.e., the overall analyte level) may be calculated as a weighted average of glucose values generated for all SAs using normalized weights wₙ_{ₐ}, where the sum of the normalized weights wₙ is 1. For example, the final glucose level (GLU) may be calculated as follows:

$$GLU = {\sum }_{a = 1}^{N} {w}_{{n}_{a}} \times {GLU}_{a} where {\sum }_{a = 1}^{N} {w}_{{n}_{a}} = 1 .$$

where GLUₐ is a glucose value generated by a certain ME group in a certain SA and N is the number of ME groups or the number of SAs.

The uniformity of the weighting may be controlled through the normalization process by the exponent k. For example, when k=0, the weighting is uniform (ωₙ=1/N). As k increases, weighting becomes 0 for all areas but the one with the highest unnormalized weight. In one example, the weight wₙ_{ₐ} can be calculated as follows:

$${ω}_{{n}_{a}} = \sqrt[k]{\frac{{ω}_{{un}_{a}}^{k}}{{\sum }_{a = 1}^{N} {ω}_{{un}_{a}}^{k}}}$$

### Here,

$${ω}_{{un}_{a}}^{k}$$

corresponds to an unnormalized weight wᵤₙ_{ₐ} modified by the exponent k.

In some aspects, the unnormalized weight wᵤₙ_{ₐ} may be composed of a weighting coming from an independent quality assessment of the data, ω_{Q}_{ₐ}, and a weighting that comes from the distribution of the glucose values across the different areas, ω_{G}_{ₐ}. For example, the unnormalized weight wᵤₙ_{ₐ} may be calculated as follows:

$${ω}_{{un}_{a}}^{k} = {ω}_{{Q}_{a}} ( M S P , M E P , MCP , Ambient Light , Blinding , etc ) \times {ω}_{{G}_{a}} ( \vec{GLU} )$$

In some aspects, MSP is a metric for real time assessment of sensor performance (MSP), which is explained in U.S. Pat. No. 10,869,624, which is hereby incorporated by reference in its entirety. In some aspects, MEP is a metric for electronic performance (MEP), which is explained in U.S. Pat. No. 11,701,038, which is hereby incorporated by reference in its entirety.

In one example, the weighting ω_{Q}_{ₐ} coming from the independent quality assessment of the data may be calculated as follows:

$${ω}_{{Q}_{a}} = {ω}_{{MSP}_{a}} \times {ω}_{{MEP}_{a}} \times {ω}_{{MCP}_{a}} \times {ω}_{{Blinding}_{a}}$$

$${ω}_{{Q}_{a}} = M S {P}_{a} \times M E {P}_{a} \times {MCP}_{a} \times ( 1 - {is\_Blinded}_{a} )$$

$$where ( 1 - {is\_Blinded}_{a} ) = ( 1 - {is\_AmbientLight}_{a} ) \times ( 1 - {is\_spike}_{a} ) \times ( 1 - {is\_jump}_{a} ) \times … .$$

In another example, the weighting ω_{Q}_{ₐ} coming from the independent quality assessment of the data may be calculated as follows:

$${ω}_{{Q}_{a}} = ( \frac{M S {P}_{a} - \text{.35}}{\text{.65}} ) \times ( \frac{M E {P}_{a} - \text{.35}}{\text{.65}} ) \times ( \frac{{MCP}_{a} - \text{.35}}{\text{.65}} ) \times max ( 1 , 1 - \frac{sigOff - {sigOff}_{QC}}{AmbientLight\_Threshold} ) \times ( 1 - \frac{max ( \vert {GLU}_{a} ( {t}_{n} ) - {GLU}_{a} ( {t}_{n - 1} ) \vert , \vert {GLU}_{a} ( {t}_{n + 1} ) - {GLU}_{a} ( {t}_{n} ) \vert}{Spike\_Threshold} ) \times … .$$

In one example, the weighting ω_{G}_{ₐ} that comes from the distribution of the glucose values across the different area may be calculated as follows:

$$If {\sum }_{n = a}^{N} ( {ω}_{{Q}_{n}} > 0 ) > 2 , {ω}_{{G}_{a}} = max ( 1 , {( 1 - {( \frac{{GLU}_{a} - CT}{cS} )}^{2} )}^{2} )$$

$$If {\sum }_{n = a}^{N} ( {ω}_{{Q}_{n}} > 0 ) \leq 2 , {ω}_{{G}_{a}} = \frac{1}{N}$$

Here, CT is a central tendency such as mean, median, bi-mean or weighted mean, S is a measure of spread such SD, IQR, or IDR, c is a parameter that controls how quickly the weights roll off. According to the above formula, when an SA's glucose value differs from the central tendency by more than cS, its weight will go to 0 (considered a complete outlier). In some aspects, 1<c<2. Note that the weighted mean can be used by assuming uniform weighting for CT and then iteratively calculating new weights using the weights from the last iteration for CT.

In another example, the weighting ω_{G}_{ₐ} that comes from the distribution of the glucose values across the different areas may be calculated as follows:

$$If {\sum }_{n = a}^{N} ( {ω}_{{Q}_{n}} > 0 ) > 3 {ω}_{{G}_{a}} = max ( 1 , {( 1 - min ( {( \frac{{GLU}_{a} - {GLU}_{min + 1}}{cS} )}^{2} , {( \frac{{GLU}_{a} - {GLU}_{max - 1}}{cS} )}^{2} ) )}^{2} )$$

$$If {\sum }_{n = a}^{N} ( {ω}_{{Q}_{n}} > 0 ) > 2 , {ω}_{{G}_{a}} = max ( 1 , {( 1 - {( \frac{{GLU}_{a} - CT}{cS} )}^{2} )}^{2} )$$

$$If {\sum }_{n = a}^{N} ( {ω}_{{Q}_{n}} > 0 ) \leq 2 , {ω}_{{G}_{a}} = \frac{1}{N}$$

Here, CT is a central tendency such as mean, median, bi-mean or weighted mean, S is a measure of spread such SD, IQR, or IDR, c is a parameter that controls how quickly the weights roll off. According to the above formula, when an SA's glucose value differs from the central tendency by more than cS, its weight will go to 0 (considered a complete outlier). In some aspects, 1<c<2. Note that the weighted mean can be used by assuming uniform weighting for CT and then iteratively calculating new weights using the weights from the last iteration for CT.

As explained above, in some aspects, the divergence of the analyte values generated for a certain SA with respect to the analyte values generated for other SAs is calculated based on the Mahalanobis distance metric. However, in other aspects, the divergence of analyte values can be calculated as follows:

$${Dev}_{Area} ( {t}_{n} ) = \sqrt{\frac{1}{Na} {\sum }_{a = 1}^{{N}_{a}} {( {GLU}_{a} ( {t}_{n} ) - CT ( {t}_{n} ) )}^{2}}$$

Dev_{Area}(tₙ) is a value indicating the divergence of analyte values generated for a certain SA at time=tₙ, Nₐ is the number of different SAs, GLUₐ(tₙ) is a Glucose level measured by a ME group in a certain SA, and CT is a central tendency of analyte values generated for all SAs (or all SAs excluding the certain SA). In some aspects,

$$CT ( {t}_{n} ) = {\sum }_{a = 1}^{{N}_{a}} {{GLU}_{a} ( {t}_{n} )}^{2} .$$

In some aspects, the divergence of analyte values may be calculated not only per SA but also per time. In such aspects, the divergence of analyte values can be calculated as follows:

$${Dev}_{Area , Time} ( {t}_{m} ) = \sqrt{\frac{1}{L} {\sum }_{n = m}^{m + 1 - L} {\sum }_{a = 1}^{{N}_{a}} {{ω}_{{Q}_{a , n}} ( {GLU}_{a} ( {t}_{n} ) - CT ( {t}_{n} ) )}^{2}}$$

Dev_{Area,Time}(tₘ) is a value indicating the divergence of analyte values generated for a certain SA at time=tₘ, Nₐ is the number of different SAs, GLUₐ(tₙ) is a Glucose level measured by a ME group in a certain SA, CT is a central tendency of analyte values generated for all SAs (or all SAs excluding the certain SA), and 1-L is the number of time intervals for measuring the analyte values.

As explained above, in case the analyte sensor 102 (or the analyte sensing device 352) includes more than one ME groups in a certain SA of the device, multiple sets of analyte values are generated. However, there may be a scenario where a set of analyte values generated for a certain SA is diverged from other sets of analyte values, and thus the diverged set of analyte values constitutes an “outlier” or low quality measurements. In this scenario, in calculating the overall analyte level, it may be beneficial to “de-weight” or exclude such outlier/low quality measurements. According to some aspects of this disclosure, such outlier/low quality measurements is detected/identified using a distribution of all analyte values and the contribution of outlier/low quality measurements towards the overall analyte level is adjusted, thereby improving the accuracy of determining the overall analyte level.

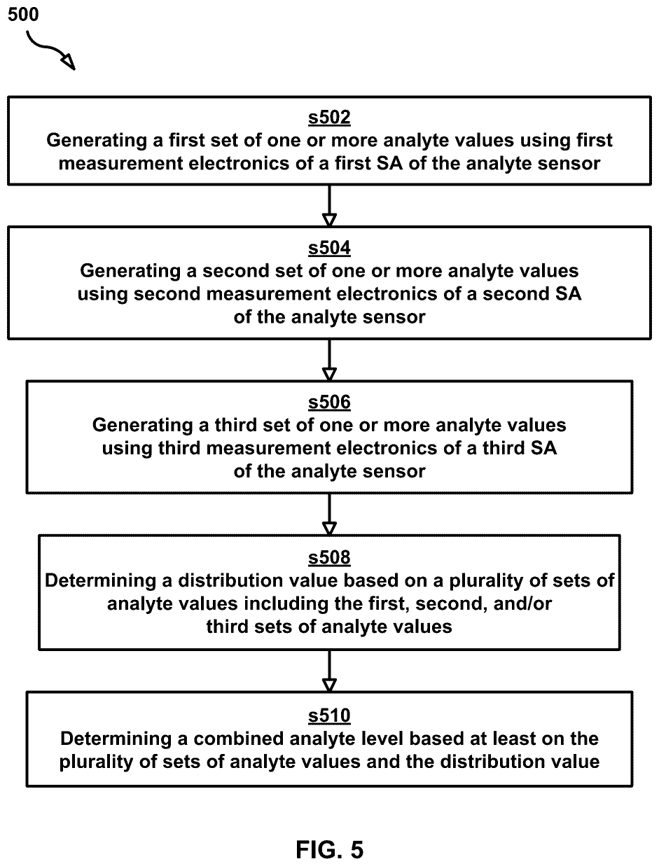

FIG. 5 shows a process 500 performed by the analyte monitoring system 100 (e.g., the analyte sensor 102, transceiver 104, and/or user device 106 of the analyte monitoring system 100) according to some aspects. In some aspects, the transceiver 104 may perform one or more steps of the process 500, and, in some alternative aspects, the analyte sensor 102 or the user device 106 may performed one or more steps of the process 500.

In some aspects, as shown in FIG. 5, the process 500 may include a step 502. The step 502 may include generating a first set 322 of one or more analyte values using first measurement electronics 302 of a first sensing area (SA) of the analyte sensor 102. In some aspects, the step 502 may, for example, be performed by the transceiver 104 using measurements generated by the measurement electronics 302 of the first SA of the analyte sensor 102 and conveyed from the analyte sensor 102. In some alternative aspects, step 502 may be performed by the user device 106 using measurements generated by the measurement electronics 302 of the first SA of the analyte sensor 102 and conveyed from the analyte sensor 102. In some further alternative aspects, step 502 may be performed by the analyte sensor 102.

In some aspects, as shown in FIG. 5, the process 500 may include a step 504. The step 504 may include generating a second set 324 of one or more analyte values using second measurement electronics 304 of a second SA of the analyte sensor 102. In some aspects, step 504 may, for example, be performed by the transceiver 104 using measurements generated by the measurement electronics 304 of the second SA of the analyte sensor 102 and conveyed from the analyte sensor 102. In some alternative aspects, step 504 may be performed by the user device 106 using measurements generated by the measurement electronics 304 of the second SA of the analyte sensor 102 and conveyed from the analyte sensor 102. In some further alternative aspects, step 504 may be performed by the analyte sensor 102.

In some aspects, as shown in FIG. 5, the process 500 may include a step 506. The step 506 may include generating a third set 326 of one or more analyte values using third measurement electronics 306 of a third SA of the analyte sensor 102. In some aspects, step 506 may, for example, be performed by the transceiver 104 using measurements generated by the measurement electronics 306 of the third SA of the analyte sensor 102 and conveyed from the analyte sensor 102. In some alternative aspects, step 506 may be performed by the user device 106 using measurements generated by the measurement electronics 306 of the third SA of the analyte sensor 102 and conveyed from the analyte sensor 102. In some further alternative aspects, step 506 may be performed by the analyte sensor 102. Although FIG. 5 shows that step 506 is performed after the step 504 and that step 504 is performed after step 502, in some alternative aspects, the step 502, 504, and 506 may be formed in a different order or simultaneously.

In some aspects, although not shown in FIG. 5, the process 500 may include an optional step of generating a fourth set 328 of one or more analyte values using fourth measurement electronics 308 of a fourth SA of the analyte sensor 102. In some aspects, the process 500 may include optional steps of generating additional sets of one or more analyte values using additional measurement electronics of additional SAs of the analyte sensor 102.

In some aspects, as shown in FIG. 5, the process 500 may include a step 508. The step 508 may include determining a distribution value based on a plurality of sets of analyte values including the first, second, and/or third sets 322, 324, and/or 326 of analyte values. In some aspects, the plurality of sets of analyte values may include the fourth set 328 of analyte values. In some aspects, the distribution value may indicate a distribution of the plurality of sets of analyte values. In some aspects, as shown in FIG. 5, the process 500 may include a step 510 of determining a combined analyte level based at least on the plurality of sets of analyte values and the distribution value.

In some aspects, the first set 322 of analyte values may include a plurality of analyte values generated for different instances of time using the first measurement electronics 302 of the first SA of the analyte sensor 102, the second set 324 of analyte values includes a plurality of analyte values generated for the different instances of time using the second measurement electronics 304 of the second SA of the analyte sensor 102, and the third set 326 of analyte values includes a plurality of analyte values generated for the different instances of time using the third measurement electronics 306 of the third SA of the analyte sensor 102. In some aspects, the fourth set 328 of analyte values may include a plurality of analyte values generated for different instances of time using the fourth measurement electronics 308 of the fourth SA of the analyte sensor 102.

In some aspects, the process 500 may include determining whether the first set 322 of analyte values satisfies a condition, determining whether the second set 324 of analyte values satisfies the condition, and determining whether the third set 326 of analyte values satisfies the condition. In some aspects, the process 500 may include, based on determining that the first and second sets of analyte values do not satisfy the condition and the third set of analyte values satisfies the condition, not selecting the first and second SAs and selecting the third SA, and the combined analyte level may be determined in step 510 based on the selection of the third SA.

In some aspects, determining the distribution value may include: calculating a first tendency value representing the third set 326 of analyte values and calculating a second tendency value representing the plurality of sets of analyte values. In some aspects, whether the third set 328 of analyte values satisfies the condition may be determined based at least on the first tendency value, the second tendency value, and the distribution value.

In some aspects, the first tendency value may be one of a mean or a median of the third set 326 of analyte values, the second tendency value may be one of a mean or a median of the plurality of sets of analyte values, and the distribution value may be one of a standard deviation, an interquartile range, or an interdecile range of the plurality of sets of analyte values.

In some aspects, determining whether the third set 326 of analyte values satisfies the condition may include: calculating a comparative value based on the first tendency value, the second tendency value, and the distribution value; determining whether the comparative value is greater than a threshold value; and determining that the third set 326 of analyte values satisfies the condition based on whether the comparative value is greater than the threshold value.

In some aspects, the comparative value may be calculated as follows:

$$d = \frac{{C}_{1} \times {T}_{1} - {C}_{2} \times {T}_{2}}{{C}_{3} \times {D}_{2}}$$

where d is the comparative value, C₁ is a first constant, C₂ is a second constant, C₃ is a third constant, T₁ is the first tendency value, T₂ is the second tendency value, and D₂ is the distribution value.

In some aspects, if the third set 326 of analyte values is determined to satisfy the condition, the distribution value may be determined not based on the third set of analyte values.

In some aspects, determining the combined analyte level may include: calculating a first SA analyte level for the first SA based on the first set 322 of analyte values; determining a first weight for the first SA analyte level; calculating a second SA analyte level for the second SA based on the second set 324 of analyte values; determining a second weight for the second SA analyte level; calculating a third SA analyte level for the third SA based on the third set 326 of analyte values; and determining a third weight for the third SA analyte level. In some aspects, the combined analyte level may be determined based on the first SA analyte level, the first weight, the second SA analyte level, the second weight, the third SA analyte level, and the third weight.

In some aspects, the first weight may be determined based at least on: the first SA analyte level; a tendency value representing the plurality of sets of analyte values; and the distribution value. In some aspects, the second weight may be determined based at least on: the second SA analyte level; the tendency value; and the distribution value. In some aspects, the third weight may be determined based at least on: the third SA analyte level; the tendency value; and the distribution value.

In some aspects, the first weight may have a negative correlation with a difference between the first SA analyte level and the tendency value increases; the second weight may have a negative correlation with a difference between the second SA analyte level and the tendency value increases; and the third weight may have a negative correlation with a difference between the third SA analyte level and the tendency value increases.

In some aspects, the tendency value may be one of a mean or a median of the plurality of sets of analyte values, and the distribution value may be one of a standard deviation, an interquartile range, or an interdecile range of the plurality of sets of analyte values.

In some aspects, the first weight may equal

$$f ( \frac{{C}_{1} \times {AL}_{SA 1} - {C}_{2} \times CT}{{C}_{3} \times S} ) ,$$

the second weight may equal

$$f ( \frac{{C}_{1} \times {AL}_{SA 2} - {C}_{2} \times CT}{{C}_{3} \times S} ) ,$$

and the third weight may equal

$$f ( \frac{{C}_{1} \times {AL}_{SA 3} - {C}_{2} \times CT}{{C}_{3} \times S} ) ,$$

where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

### Summary of Embodiments

A1. A method (500) performed by an analyte monitoring system (100) comprising an analyte sensor (102), the method comprising: generating (s502) a first set of one or more analyte values using first measurement electronics of a first sensing area (SA) of the analyte sensor; generating (s504) a second set of one or more analyte values using second measurement electronics of a second SA of the analyte sensor, wherein the first and second SAs are different; generating (s506) a third set of one or more analyte values using third measurement electronics of a third SA of the analyte sensor, wherein the first, second, and third SAs are different; determining (s508) a distribution value based on a plurality of sets of analyte values including the first, second, and/or third sets of analyte values, wherein the distribution value indicates a distribution of the plurality of sets of analyte values; and determining (s510) a combined analyte level based at least on the plurality of sets of analyte values and the distribution value.

A2. The analyte monitoring system of embodiment A1, wherein: the first set of analyte values includes a plurality of analyte values generated for different instances of time using the first measurement electronics of the first SA of the analyte sensor, the second set of analyte values includes a plurality of analyte values generated for the different instances of time using the second measurement electronics of the second SA of the analyte sensor, and the third set of analyte values includes a plurality of analyte values generated for the different instances of time using the third measurement electronics of the third SA of the analyte sensor.

A3. The method of embodiment A1 or A2, comprising: determining whether the first set of analyte values satisfies a condition; determining whether the second set of analyte values satisfies the condition; determining whether the third set of analyte values satisfies the condition; and, based on determining that the first and second sets of analyte values do not satisfy the condition and the third set of analyte values satisfies the condition, not selecting the first and second SAs and selecting the third SA, wherein the combined analyte level is determined based on the selection of the third SA.

A4. The method of embodiment A3, wherein: the analyte sensor comprises a plurality of SAs including the first, second, and third SAs; the method comprises generating the plurality of set of analyte values using measurement electronics of the plurality of SAs of the analyte sensor; determining the distribution value comprises: calculating a first tendency value representing the third set of analyte values, and calculating a second tendency value representing the plurality of sets of analyte values, and whether the third set of analyte values satisfies the condition is determined based at least on the first tendency value, the second tendency value, and the distribution value.

A5. The method of embodiment A4, wherein: the first tendency value is one of a mean or a median of the third set of analyte values, the second tendency value is one of a mean or a median of the plurality of sets of analyte values, and the distribution value is one of a standard deviation, an interquartile range, or an interdecile range of the plurality of sets of analyte values.

A6. The method of embodiment A4 or A5, wherein determining whether the third set of analyte values satisfies the condition comprises: calculating a comparative value based on the first tendency value, the second tendency value, and the distribution value; determining whether the comparative value is greater than a threshold value; and determining that the third set of analyte values satisfies the condition based on whether the comparative value is greater than the threshold value.

A7. The method of embodiment A6, wherein the comparative value is calculated as follows:

$$d = \frac{{C}_{1} \times {T}_{1} - {C}_{2} \times {T}_{2}}{{C}_{3} \times {D}_{2}}$$

where d is the comparative value, C₁ is a first constant, C₂ is a second constant, C₃ is a third constant, T₁ is the first tendency value, T₂ is the second tendency value, and D₂ is the distribution value.

A8. The method of any one of embodiments A3-A7, wherein, if the third set of analyte values is determined to satisfy the condition, the distribution value is determined not based on the third set of analyte values.

A9. The method of any one of embodiments A1-A7, wherein determining the combined analyte level comprises: calculating a first SA analyte level for the first SA based on the first set of analyte values; determining a first weight for the first SA analyte level; calculating a second SA analyte level for the second SA based on the second set of analyte values; determining a second weight for the second SA analyte level; calculating a third SA analyte level for the third SA based on the third set of analyte values; and determining a third weight for the third SA analyte level; wherein the combined analyte level is determined based on the first SA analyte level, the first weight, the second SA analyte level, the second weight, the third SA analyte level, and the third weight.

A10. The method of embodiment A9, wherein: the first weight is determined based at least on: the first SA analyte level; a tendency value representing the plurality of sets of analyte values; and the distribution value, the second weight is determined based at least on: the second SA analyte level; the tendency value; and the distribution value, and the third weight is determined based at least on: the third SA analyte level; the tendency value; and the distribution value.

A11. The method of embodiment A10, wherein: the first weight has a negative correlation with a difference between the first SA analyte level and the tendency value increases; the second weight has a negative correlation with a difference between the second SA analyte level and the tendency value increases; and the third weight has a negative correlation with a difference between the third SA analyte level and the tendency value increases.

A12. The method of embodiment A10 or A11, wherein: the tendency value is one of a mean or a median of the plurality of sets of analyte values, and the distribution value is one of a standard deviation, an interquartile range, or an interdecile range of the plurality of sets of analyte values.

A13. The method of any one of embodiments A10-A12, wherein;

$$the first weight = f ( \frac{{C}_{1} \times {AL}_{SA 1} - {C}_{2} \times CT}{{C}_{3} \times S} ) ,$$

$$the second weight = f ( \frac{{C}_{1} \times {AL}_{SA 2} - {C}_{2} \times CT}{{C}_{3} \times S} ) ,$$

$$and the third weight = f ( \frac{{C}_{1} \times {AL}_{SA 3} - {C}_{2} \times CT}{{C}_{3} \times S} ) ,$$

where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

B1. An analyte monitoring system (100) comprising: an analyte sensor (102); a user device (106), wherein the analyte monitoring system is configured to: generate (502) a first set of one or more analyte values using first measurement electronics of a first sensing area (SA) of the analyte sensor; generate (504) a second set of one or more analyte values using second measurement electronics of a second SA of the analyte sensor, wherein the first and second SAs are different; generate (506) a third set of one or more analyte values using third measurement electronics of a third SA of the analyte sensor, wherein the first, second, and third SAs are different; determine (508) a distribution value based on a plurality of sets of analyte values including the first, second, and/or third sets of analyte values, wherein the distribution value indicates a distribution of the plurality of sets of analyte values; and determine (510) a combined analyte level based at least on the plurality of sets of analyte values and the distribution value.

B2. The analyte monitoring system of embodiment B1, wherein: the first set of analyte values includes a plurality of analyte values generated for different instances of time using the first measurement electronics of the first SA of the analyte sensor, the second set of analyte values includes a plurality of analyte values generated for the different instances of time using the second measurement electronics of the second SA of the analyte sensor, and the third set of analyte values includes a plurality of analyte values generated for the different instances of time using the third measurement electronics of the third SA of the analyte sensor.

B3. The analyte monitoring system of embodiment B1 or B2, wherein the analyte monitoring system is configured to: determine whether the first set of analyte values satisfies a condition; determine whether the second set of analyte values satisfies the condition; determine whether the third set of analyte values satisfies the condition; and, based on determining that the first and second sets of analyte values do not satisfy the condition and the third set of analyte values satisfies the condition, not select the first and second SAs and selecting the third SA, wherein the combined analyte level is determined based on the selection of the third SA.

B4. The analyte monitoring system of embodiment B3, wherein: the analyte sensor comprises a plurality of SAs including the first, second, and third SAs; the analyte monitoring system is configured to generate the plurality of set of analyte values using measurement electronics of the plurality of SAs of the analyte sensor; determining the distribution value comprises: calculating a first tendency value representing the third set of analyte values, and calculating a second tendency value representing the plurality of sets of analyte values; and whether the third set of analyte values satisfies the condition is determined based at least on the first tendency value, the second tendency value, and the distribution value.

B5. The analyte monitoring system of embodiment B4, wherein: the first tendency value is one of a mean or a median of the third set of analyte values, the second tendency value is one of a mean or a median of the plurality of sets of analyte values, and the distribution value is one of a standard deviation, an interquartile range, or an interdecile range of the plurality of sets of analyte values.

B6. The analyte monitoring system of embodiment B4 or B5, wherein determining whether the third set of analyte values satisfies the condition comprises: calculating a comparative value based on the first tendency value, the second tendency value, and the distribution value; determining whether the comparative value is greater than a threshold value; and determining that the third set of analyte values satisfies the condition based on whether the comparative value is greater than the threshold value.

B7. The analyte monitoring system of embodiment B6, wherein the comparative value is calculated as follows:

$$d = \frac{{C}_{1} \times {T}_{1} - {C}_{2} \times {T}_{2}}{{C}_{3} \times {D}_{2}}$$

where d is the comparative value, C₁ is a first constant, C₂ is a second constant, C₃ is a third constant, T₁ is the first tendency value, T₂ is the second tendency value, and D₂ is the distribution value.

B8. The analyte monitoring system of any one of embodiments B3-B7, wherein, if the third set of analyte values is determined to satisfy the condition, the distribution value is determined not based on the third set of analyte values.

B9. The analyte monitoring system of any one of embodiments B1-B7, wherein determining the combined analyte level comprises: calculating a first SA analyte level for the first SA based on the first set of analyte values; determining a first weight for the first SA analyte level; calculating a second SA analyte level for the second SA based on the second set of analyte values; determining a second weight for the second SA analyte level; calculating a third SA analyte level for the third SA based on the third set of analyte values; and determining a third weight for the third SA analyte level; wherein the combined analyte level is determined based on the first SA analyte level, the first weight, the second SA analyte level, the second weight, the third SA analyte level, and the third weight.

B10. The analyte monitoring system of embodiment B9, wherein: the first weight is determined based at least on: the first SA analyte level, a tendency value representing the plurality of sets of analyte values, and the distribution value; the second weight is determined based at least on: the second SA analyte level, the tendency value, and the distribution value; and the third weight is determined based at least on: the third SA analyte level, the tendency value, and the distribution value.

B11. The analyte monitoring system of embodiment B10, wherein: the first weight has a negative correlation with a difference between the first SA analyte level and the tendency value increases; the second weight has a negative correlation with a difference between the second SA analyte level and the tendency value increases; and the third weight has a negative correlation with a difference between the third SA analyte level and the tendency value increases.

B12. The analyte monitoring system of embodiment B10 or B11, wherein: the tendency value is one of a mean or a median of the plurality of sets of analyte values, and the distribution value is one of a standard deviation, an interquartile range, or an interdecile range of the plurality of sets of analyte values.

B13. The analyte monitoring system of any one of embodiments B10-B12, wherein;

$$the first weight = f ( \frac{{C}_{1} \times {AL}_{SA 1} - {C}_{2} \times CT}{{C}_{3} \times S} ) ,$$

$$the second weight = f ( \frac{{C}_{1} \times {AL}_{SA 2} - {C}_{2} \times CT}{{C}_{3} \times S} ) ,$$

$$and the third weight = f ( \frac{{C}_{1} \times {AL}_{SA 3} - {C}_{2} \times CT}{{C}_{3} \times S} ) ,$$

where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

While various aspects are described herein, it should be understood that they have been presented by way of example only, and not limitation. Thus, the breadth and scope of this disclosure should not be limited by any of the above-described exemplary aspects. Moreover, any combination of the above-described elements in all possible variations thereof is encompassed by the disclosure unless otherwise indicated herein or otherwise clearly contradicted by context.

As used herein transmitting a message “to” or “toward” an intended recipient encompasses transmitting the message directly to the intended recipient or transmitting the message indirectly to the intended recipient (i.e., one or more other nodes are used to relay the message from the source node to the intended recipient). Likewise, as used herein receiving a message “from” a sender encompasses receiving the message directly from the sender or indirectly from the sender (i.e., one or more nodes are used to relay the message from the sender to the receiving node). Further, as used herein “a” means “at least one” or “one or more.”

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

What is claimed is:

1. A method performed by an analyte monitoring system comprising an analyte sensor, the method comprising:
generating a first set of one or more analyte values using first measurement electronics of a first sensing area (SA) of the analyte sensor;
generating a second set of one or more analyte values using second measurement electronics of a second SA of the analyte sensor, wherein the first and second SAs are different;
generating a third set of one or more analyte values using third measurement electronics of a third SA of the analyte sensor, wherein the first, second, and third SAs are different;
determining a distribution value based on a plurality of sets of analyte values including the first, second, and/or third sets of analyte values, wherein the distribution value indicates a distribution of the plurality of sets of analyte values; and
determining a combined analyte level based at least on the plurality of sets of analyte values and the distribution value.

2. The analyte monitoring system of claim 1, wherein
the first set of analyte values includes a plurality of analyte values generated for different instances of time using the first measurement electronics of the first SA of the analyte sensor,
the second set of analyte values includes a plurality of analyte values generated for the different instances of time using the second measurement electronics of the second SA of the analyte sensor, and
the third set of analyte values includes a plurality of analyte values generated for the different instances of time using the third measurement electronics of the third SA of the analyte sensor.

3. The method of claim 1, comprising:
determining whether the first set of analyte values satisfies a condition;
determining whether the second set of analyte values satisfies the condition;
determining whether the third set of analyte values satisfies the condition; and
based on determining that the first and second sets of analyte values do not satisfy the condition and the third set of analyte values satisfies the condition, not selecting the first and second SAs and selecting the third SA, wherein the combined analyte level is determined based on the selection of the third SA.

4. The method of claim 3, wherein:
the analyte sensor comprises a plurality of SAs including the first, second, and third SAs,
the method comprises generating the plurality of set of analyte values using measurement electronics of the plurality of SAs of the analyte sensor,
determining the distribution value comprises:
calculating a first tendency value representing the third set of analyte values, and
calculating a second tendency value representing the plurality of sets of analyte values, and
whether the third set of analyte values satisfies the condition is determined based at least on the first tendency value, the second tendency value, and the distribution value.

5. The method of claim 4, wherein:
the first tendency value is one of a mean or a median of the third set of analyte values,
the second tendency value is one of a mean or a median of the plurality of sets of analyte values, and
the distribution value is one of a standard deviation, an interquartile range, or an interdecile range of the plurality of sets of analyte values.

6. The method of claim 4, wherein determining whether the third set of analyte values satisfies the condition comprises:
calculating a comparative value based on the first tendency value, the second tendency value, and the distribution value;
determining whether the comparative value is greater than a threshold value; and
determining that the third set of analyte values satisfies the condition based on whether the comparative value is greater than the threshold value.

7. The method of claim 6, wherein the comparative value is calculated as follows:
$$d = \frac{{C}_{1} \times {T}_{1} - {C}_{2} \times {T}_{2}}{{C}_{3} \times {D}_{2}}$$
where d is the comparative value, C₁ is a first constant, C₂ is a second constant, C₃ is a third constant, T₁ is the first tendency value, T₂ is the second tendency value, and D₂ is the distribution value.

8. The method of claim 3, wherein, if the third set of analyte values is determined to satisfy the condition, the distribution value is determined not based on the third set of analyte values.

9. The method of claim 1, wherein determining the combined analyte level comprises:
calculating a first SA analyte level for the first SA based on the first set of analyte values;
determining a first weight for the first SA analyte level;
calculating a second SA analyte level for the second SA based on the second set of analyte values;
determining a second weight for the second SA analyte level;
calculating a third SA analyte level for the third SA based on the third set of analyte values; and
determining a third weight for the third SA analyte level;
wherein the combined analyte level is determined based on the first SA analyte level, the first weight, the second SA analyte level, the second weight, the third SA analyte level, and the third weight.

10. The method of claim 9, wherein:
the first weight is determined based at least on:
the first SA analyte level;
a tendency value representing the plurality of sets of analyte values; and
the distribution value,
the second weight is determined based at least on:
the second SA analyte level;
the tendency value; and
the distribution value, and
the third weight is determined based at least on:
the third SA analyte level;
the tendency value; and
the distribution value.

11. The method of claim 10, wherein:
the first weight has a negative correlation with a difference between the first SA analyte level and the tendency value increases;
the second weight has a negative correlation with a difference between the second SA analyte level and the tendency value increases; and
the third weight has a negative correlation with a difference between the third SA analyte level and the tendency value increases.

12. The method of claim 10, wherein:
the tendency value is one of a mean or a median of the plurality of sets of analyte values, and
the distribution value is one of a standard deviation, an interquartile range, or an interdecile range of the plurality of sets of analyte values.

13. The method of claim 10, wherein;
$$the first weight = f ( \frac{{C}_{1} \times {AL}_{SA 1} - {C}_{2} \times CT}{{C}_{3} \times S} ) ,$$
$$the second weight = f ( \frac{{C}_{1} \times {AL}_{SA 2} - {C}_{2} \times CT}{{C}_{3} \times S} ) , and$$
$$the third weight = f ( \frac{{C}_{1} \times {AL}_{SA 3} - {C}_{2} \times CT}{{C}_{3} \times S} ) ,$$
where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.

14. An analyte monitoring system comprising:
an analyte sensor;
a user device, wherein the analyte monitoring system is configured to:
generate a first set of one or more analyte values using first measurement electronics of a first sensing area (SA) of the analyte sensor;
generate a second set of one or more analyte values using second measurement electronics of a second SA of the analyte sensor, wherein the first and second SAs are different;
generate a third set of one or more analyte values using third measurement electronics of a third SA of the analyte sensor, wherein the first, second, and third SAs are different;
determine a distribution value based on a plurality of sets of analyte values including the first, second, and/or third sets of analyte values, wherein the distribution value indicates a distribution of the plurality of sets of analyte values; and
determine a combined analyte level based at least on the plurality of sets of analyte values and the distribution value.

15. The analyte monitoring system of claim 14, wherein
the first set of analyte values includes a plurality of analyte values generated for different instances of time using the first measurement electronics of the first SA of the analyte sensor,
the second set of analyte values includes a plurality of analyte values generated for the different instances of time using the second measurement electronics of the second SA of the analyte sensor, and
the third set of analyte values includes a plurality of analyte values generated for the different instances of time using the third measurement electronics of the third SA of the analyte sensor.

16. The analyte monitoring system of claim 14, wherein the analyte monitoring system is configured to:
determine whether the first set of analyte values satisfies a condition;
determine whether the second set of analyte values satisfies the condition;
determine whether the third set of analyte values satisfies the condition; and
based on determining that the first and second sets of analyte values do not satisfy the condition and the third set of analyte values satisfies the condition, not select the first and second SAs and selecting the third SA, wherein the combined analyte level is determined based on the selection of the third SA.

17. The analyte monitoring system of claim 16, wherein:
the analyte sensor comprises a plurality of SAs including the first, second, and third SAs,
the analyte monitoring system is configured to generate the plurality of set of analyte values using measurement electronics of the plurality of SAs of the analyte sensor,
determining the distribution value comprises:
calculating a first tendency value representing the third set of analyte values, and
calculating a second tendency value representing the plurality of sets of analyte values, and
whether the third set of analyte values satisfies the condition is determined based at least on the first tendency value, the second tendency value, and the distribution value.

18. The analyte monitoring system of claim 17, wherein:
the first tendency value is one of a mean or a median of the third set of analyte values,
the second tendency value is one of a mean or a median of the plurality of sets of analyte values, and
the distribution value is one of a standard deviation, an interquartile range, or an interdecile range of the plurality of sets of analyte values.

19. The analyte monitoring system of claim 17, wherein determining whether the third set of analyte values satisfies the condition comprises:
calculating a comparative value based on the first tendency value, the second tendency value, and the distribution value;
determining whether the comparative value is greater than a threshold value; and
determining that the third set of analyte values satisfies the condition based on whether the comparative value is greater than the threshold value.

20. The analyte monitoring system of claim 19, wherein the comparative value is calculated as follows:
$$d = \frac{{C}_{1} \times {T}_{1} - {C}_{2} \times {T}_{2}}{{C}_{3} \times {D}_{2}}$$
where d is the comparative value, C₁ is a first constant, C₂ is a second constant, C₃ is a third constant, T₁ is the first tendency value, T₂ is the second tendency value, and D₂ is the distribution value.

21. The analyte monitoring system of claim 16, wherein, if the third set of analyte values is determined to satisfy the condition, the distribution value is determined not based on the third set of analyte values.

22. The analyte monitoring system of claim 14, wherein determining the combined analyte level comprises:
calculating a first SA analyte level for the first SA based on the first set of analyte values;
determining a first weight for the first SA analyte level;
calculating a second SA analyte level for the second SA based on the second set of analyte values;
determining a second weight for the second SA analyte level;
calculating a third SA analyte level for the third SA based on the third set of analyte values; and
determining a third weight for the third SA analyte level;
wherein the combined analyte level is determined based on the first SA analyte level, the first weight, the second SA analyte level, the second weight, the third SA analyte level, and the third weight.

23. The analyte monitoring system of claim 22, wherein:
the first weight is determined based at least on:
the first SA analyte level;
a tendency value representing the plurality of sets of analyte values; and
the distribution value,
the second weight is determined based at least on:
the second SA analyte level;
the tendency value; and
the distribution value, and
the third weight is determined based at least on:
the third SA analyte level;
the tendency value; and
the distribution value.

24. The analyte monitoring system of claim 23, wherein:
the first weight has a negative correlation with a difference between the first SA analyte level and the tendency value increases;
the second weight has a negative correlation with a difference between the second SA analyte level and the tendency value increases; and
the third weight has a negative correlation with a difference between the third SA analyte level and the tendency value increases.

25. The analyte monitoring system of claim 23, wherein:
the tendency value is one of a mean or a median of the plurality of sets of analyte values, and
the distribution value is one of a standard deviation, an interquartile range, or an interdecile range of the plurality of sets of analyte values.

26. The analyte monitoring system of claim 23, wherein;
$$the first weight = f ( \frac{{C}_{1} \times {AL}_{SA 1} - {C}_{2} \times CT}{{C}_{3} \times S} ) ,$$
$$the second weight = f ( \frac{{C}_{1} \times {AL}_{SA 2} - {C}_{2} \times CT}{{C}_{3} \times S} ) , and$$
$$the third weight = f ( \frac{{C}_{1} \times {AL}_{SA 3} - {C}_{2} \times CT}{{C}_{3} \times S} ) ,$$
where C₁ is a constant, AL_{SA1} is the first SA analyte level, AL_{SA2} is the second SA analyte level, AL_{SA3} is the third SA analyte level, C₂ is a constant, CT is the tendency value, C₃ is a constant determining how quickly the first, second, and third weights change depending on a difference between an SA analyte level and the tendency value, and S is the distribution value.
